(19) United States
(12) Patent Application Publication
(10) Pub. No.: US 2002/0082307 A1
(43) Pub. Date: Jun. 27, 2002

(54) COMPOSITIONS HAVING IMPROVED STABILITY

(76) Inventors: DOUGLAS JOSEPH DOBROZSI, LOVELAND, OH (US); FRANCIS JOSEPH DAVID BEALIN-KELLY, WALTON ON THAMES (GB); JAYANT EKNATH KHANOLKAR, SURBITON (GB); BRIAN JAMES ROBBINS, STAINES (GB); RICHARD MATTHEW CHARLES SUTTON, CAMBERLEY (GB)

Correspondence Address: THE PROCTER & GAMBLE COMPANY, PATENT DIVISION, HEALTH CARE RESEARCH CENTER, 8340 MASON-MONTGOMERY ROAD, MASON, OH, 45040, US

(21) Appl. No.: 09/467,333

(22) Filed: Dec. 20, 1999

Related U.S. Application Data
(60) Provisional application No. 60/156,540, filed on Sep. 29, 1999.
(60) Provisional application No. 60/115,378, filed on Jan. 11, 1999.

(51) Int. Cl. A61K 9/48; A61K 9/20; A61K 47/00; A61K 47/30; A61K 47/32; A61K 47/34

(52) U.S. Cl. 514/772; 424/464; 424/451

(57) ABSTRACT

The present invention pertains to liquid compositions having improved delivery of pharmaceutical actives. These compositions comprise pharmaceutical actives, solvent and a reducing agent. These compositions may take the form of liquid elixirs placed into the mouth by liquid-filled drops, metered liquid dosing devices, atomizers and liquid-releasing, edible capsules.

### TECHNICAL FIELD

The present invention pertains to improved stability of liquid compositions that deliver pharmaceutical active ingredients. These compositions have exceptional stability when used in various product forms including liquid elixirs placed into the mouth and eventually swallowed, or can be delivered via liquid-filled lozenges, metered liquid dosing devices, atomizers and liquid-releasing, edible capsules. Such compositions are particularly useful for treating symptoms associated with respiratory illnesses.

### BACKGROUND OF THE INVENTION

Routes for delivering pharmaceutical actives include delivering actives by intranasal, pulmonary, buccal, sublingual, transdermal, and rectal administration. These routes tend to be used for avoiding first-pass metabolism of drugs that are swallowed. “First past metabolism” refers to the arrangement and order of placement of the metabolizing enzymes within the body of a human, with respect to the path followed by substances that enter the gastrointestinal tract by swallowing, and are absorbed into the general blood circulation. Items swallowed by humans, including food, drink, and medicines, enter the stomach and from there flow into the intestine. Many of the chemicals associated with the food, drink, or medicine pass through the mucosal membranes in the gastrointestinal tract and into the blood in the mesenteric veins draining from the intestine. The blood flow from the mesenteric veins passes into the liver. Metabolizing enzymes in the mucosal membranes of the intestine and in the liver can chemically alter the nature of substances passing from the intestine, through the liver, and into the common blood circulation of the body. Since all swallowed medicines are subject to the metabolizing capacity of the intestinal mucosal membranes and the liver before entering the general blood circulation of the body, frequently only a small fraction of those substances go un-metabolized, and reach the general blood circulation

Avoiding first pass metabolism can increase the bioavailability, or blood concentrations of the administered compound. Metabolic formation of metabolites of the administered compound, however, can at the same time decrease. Where formation of metabolites from the first pass metabolism is desirable, avoiding the first pass metabolism is not preferred since it logically leads to lower amounts of the metabolite in the blood. Furthermore, the blood concentrations of the active substance can increase, leading to potential toxicity or side effects attributable to the active per se. Reducing the amount of active in the dose for avoiding toxicity, concomitantly decreases the circulating blood levels of the active metabolite. This results in loss of therapeutic affect and ultimately, benefit to the patient. In order to provide a medication that is effective and avoids unwanted side effects, the composition and its means of delivery must be modified.

Respiratory illnesses covers a broad range of ailments, including viral infections and allergic reaction to inhaled allergens. Viral infections in the upper respiratory tract of humans leads to illness usually referred to as colds, or influenza. Such an illness is quite common in the general population and can be the cause of significant discomfort and suffering. Allergen inhalation also negatively impacts a fair number in the population at the same or even at a greater degree than those having a viral infection.

There are no generally regarded effective and convenient methods for preventing viral infections or allergies. In the case of viral infections, the body's natural defense mechanisms fight the infection for a period of time normally ranging from 3 days to 2 weeks. This being the case, the most commonly employed medicines treat the uncomfortable, problematic symptoms of these respiratory ailments. These symptoms include stuffy and runny noses, soreness and inflammation in the nose and throat, fits of coughing, general aches in the body, fever, and headache. Of these symptoms, coughing in uncontrollable fits is considered by many to be the most problematic and uncomfortable. Coughing disrupts normal respiration, leading to increased headache and sore throat as well as loss of sleep to the sufferer and others living with the sufferer

The compositions used to treat the above mentioned symptoms generally fall into one of the following pharmacological classifications: antihistamines; decongestants; antitussives; expectorants; mucolytics; analgesics, antipyretic and anti-inflammatory agents. The compositions are manufactured in a number of product forms, the most common being liquid syrups and elixirs for swallowing, mouth drops and lozenges as well as inhalants and topical creams or lotions that release volatile agents that are inhaled through the nose into respiratory tract. The compositions are typically swallowed immediately, or slowly dissolved in the mouth. They typically contain actives such as guaifenesin, that aids the body in the removal of excess respiratory mucus or phlegm, diphenhydramine, that lessens the negative effects including coughing and other symptoms due to histamine produced in the body in response to the viral infection, and dextromethorphan, that acts within the part of the human brain controlling the coughing reflex. Among these actives, dextromethorphan is the most commonly used active in the world for relief of cough.

Dextromethorphan, by virtue of it's physicochemical, absorption, and bioavailability properties, is a very good candidate for increasing bioavailability via methods of administration other than swallowing. For example it has been reported in patents and pharmaceutical literature that substantial increases in bioavailability can be achieved using intranasal formulations; see H. Char et al, Nasal Deliver of 14-C dextromethorphan in Rats, Journal of Pharmaceutical Sciences 81:750, 1992.

U.S. pat. No. 4,839,176, Pankhania et al. to Boots Company, issued Jun. 13, 1989, discloses the use of bisulfites in making tablets comprising CMC that avoid degradation. U.S. pat. No. 4,474,985, Keel et al., Sep. 25, 1993 to Monsanto, discloses a process for increasing the color-free shelf life of a crude N- aminophenol. The process comprises dissolving the crude N-acetyl aminophenol in a solvent containing a reducing agent, such as meta bisulfite. U.S. pat. No. 4,478,822, issued Oct. 23, 1984 and U.S. pat. No. 4,474,752, issued Oct. 2, 1984 both to Haslam et al, and assigned to Merck & Co. claim gel compositions comprising polymers that provide gelling of the liquid when entering the body cavity. Disclosed is a group of microbiological preservatives including sodium bisulfite and sodium thiosulfate. The art know to the applicants does not demonstrate a specific chemical stabilization benefit by the inclusion of for solution or liquid-based product forms.

### SUMMARY OF THE INVENTION

What has not been realized until now is that active compounds that are combined with traditional solvents can be positively impacted when particular agents are added to the compositions. Surprisingly, adding reducing agents to a liquid composition comprising pharmaceutical actives improves the active's stability in such compositions.

The compositions of the present invention provide excellent delivery of actives to oral surfaces when in for example, a peroral product form. These compositions also demonstrate excellent shelf-life when incorporated into a variety of product forms including liquid-filled lozenges, metered liquid dosing devices, atomizers and liquidreleasing, edible capsules. Such compositions are particularly useful for treating symptoms associated with respiratory illnesses.

What has not been realized until now is that after careful and diligent research into pharmaceutic, therapeutic, and side effect properties of active compounds, compositions can be made to positively improve the therapeutic effect without increased side effects or toxicity. These compounds have improved stability in the product form selected to deliver such compositions. This benefit is achieved by adding to the active containing formulation agents that promote stability of the active in the formulation. These agents are effective in reducing and even eliminating instability due to the active's oxidation degradation pathway, thereby extending the shelf life of the compositions.

One object, therefore, of the present invention is to provide improved compositions for treating the symptoms associated with respiratory ailments, particularly minimizing fits of coughing. One particularly preferred composition is in the form of an anhydrous, hydrophilic liquids in a very stable enviroment for rapid delivery of actives including antitussives; antihistamines (including non-sedating antihistamines); decongestants; expectorants; mucolytics; analgesic, antipyretic and anti-inflammatory agents and local anesthetics for treating the symptoms of respiratory illnesses. The compositions can be dosed using a variety of product forms and, or package delivery options. The compositions of the present invention provide desired activity while minimizing potential side effects of the active compounds. It is also an objective of the subject invention to provide methods for achieving rapid transmucosal delivery of the aforementioned compositions.

### Definitions and Terms

The following are definitions of terms found in the present specification:

1. transmucosal delivery:

Refers to application of drugs to the mucosal membranes of the oral cavity, including buccal (cheek), lips, gums, palates, and tongue, with the goal of the drug passing through the skin covering these sites and entering the bloodstream.

2. therapeutic dose

Refers to the amount of the substance that when administered to a person in the proper form, will produce the desired effect within the body with minimal undesired side effects.

3. pharmaceutical active/active:

Refers to the chemical molecule which exerts the desired effect on the body, when administered in the proper amount and form.

4. active metabolites

Refers to the chemical species of the pharmaceutical active which is formed upon the active undergoing metabolism.

5. monomolecular dispersion

Refers to the fact that molecules of the active are free and unencumbered from diffusion by association in crystalline or amorphous solid forms, or poly molecular association.

6. percent solubility value

Refers to the equilibrium solubility limit or maximum solubility of a molecule in a solvent at usual room temperature, expressed as the weight percent of the molecule in the composition.

7. anhydrous solvent

Refers to solvents containing less than about 5% water.

### DETAILED DESCRIPTION OF THE INVENTION

Pharmaceutical Actives

The compositions of the present invention comprise pharmaceutical actives also referred to herein as “actives” for treating illnesses, particularly symptoms associated with respiratory ailments such as colds, influenza as well as allergy. These actives include those frequently used for treating the most problematic symptoms including a stuffy and runny nose, soreness and inflammation in the nose and throat, fits of coughing, general aches in the body, fever, and headache. In the present invention, when actives are combined with solvents, the actives obtain enhanced transmucosal delivery into the blood In the case that active metabolites contribute to the desired therapeutic effect, this enhanced delivery is achieved without appreciably lowering the level of the corresponding active metabolites. Furthermore, the level of active in the blood is maintained at a level that avoids unwanted side effects brought on by too high of levels of active in the blood.

The composition comprises a pharmaceutical active and a solvent. In a particularly preferred embodiment the solvent is a hydrophilic, water-miscible, anhydrous solvent wherein the pharmaceutical active in its un-ionized form has a percent solubility value in the solvent at ambient temperature that is equal to or greater than 0.075% and the pharmaceutical active is in its free, un-ionized form as a monomolecular dispersion in the solvent.

The preferable pharmaceutical actives of the present invention have molecular weight of less than 500 grams per mole, is capable of being ionized when in an aqueous solvent and has an octanol-water partition coefficient when in the un-ionized form of at least 100. The octanol-water partition coefficient is disclosed in A. Martin, P. Bustamante, and A. H. C. Chun, Physical Pharmacy, Fourth Edition, Lea and Febiger publishers, Philadelphia, 1993, page 237; herein incorporated by reference.

The actives that comprise compositions of the present invention include actives that fall into at least one of the following pharmacological classifications: antitussives; antihistamines; non-sedating antihistamines; decongestants; expectorants; mucolytics, analgesic, antipyretic anti-inflammatory agents, local anesthetics and mixtures thereof. References that describe the use of such actives include J. G. Hardman, The Pharmacologic Basis of Therapeutics, Ninth Edition, McGraw-Hill, New York, 1995. Among the actives that fall in these pharmacological classifications are those that are suited for absorption through mucosal tissues. These actives can be used alone or in combination with other actives not necessarily absorbed in this manner and may be formulated within existing formulation techniques.

When using actives intended for mucosal absorption, the concentration of actives in the solvent portion of the composition is preferably less than or equal to 125% of the percent solubility value, more preferably less than or equal to the percent solubility value of the pharmaceutical active. To maximize the benefits of the compositions of the present invention, the active is preferably in solution as monomolecular dispersion. The absorbed actives useful in the present invention are present in the solvent system at a level from about 0.075% to about 25.0%, preferably from about 0.28% to 10.0% by weight of the composition. It is preferred that said active is in it free, un-ionized form as a monomolecular dispersion in said solvent system. In the cases where either the salt forms or ionized forms of the drug active exist, it is preferred to use the uncharged free (non salt) form of the drug in the present invention.

Antitussives are actives of particularly use for arresting uncontrollable fits coughing. Antitussives useful in the present invention include, but, are not restricted to the group consisting of codeine, dextromethorphan, dextrorphan, diphenhydramine, hydrocodone, noscapine, oxycodone, pentoxyverine and mixtures thereof. Of these antitussives, dextromethorphan is preferred. Dextromethorphan is known to have pharmacological activity as an antitussive agent and is described in U.S. pat. No. 5,196,436, Smith; incorporated herein by reference. As used herein, “dextromethorphan” means racemethorphan, 3-methoxy-17-methylmorphinan (dl-cis-1,3,4,9,10,10a-hexahydro-6-methoxy-11-methyl-2H-10,4a-iminoethanophenanthrene and pharmaceutically-acceptable salts thereof. Compositions of the present comprising dextromethorphan preferably comprise from about 0.1% to about 9.3%, more preferably from about 0.26% to about 6.2% and most preferably from about 1.16% to about 4.6% dextromethorphan. Other safe and effective amounts of other cough/cold drug actives may be included in such dextromethorphan-containing compositions.

Antihistamines useful in the present invention include, but, are not restricted to the group consisting of acrivastine, azatadine, brompheniramine, chlorpheniramine, clemastine, cyproheptadine, dexbrompheniramine, dimenhydrinate, diphenhydramine, doxylamine, hydroxyzine, meclizine, pheninamine, phenyltoloxamine, promethazine, pyrilamine, tripelennamine, triprolidine and mixtures thereof. Non-sedating antihistamines useful in the present invention include, but, are not restricted to the group consisting of astemizole, cetirizine, ebastine, fexofenadine, loratidine, terfenadine, and mixtures thereof. Decongestants useful in the present invention include, but, are not restricted to the group consisting of phenylpropanolamine, pseudoephedrine, ephedrine, phenylephrine, oxymetazoline, and mixtures thereof Expectorants useful in the present invention include, but, are not restricted to the group consisting of ammonium chloride, guafenesin, ipecac fluid extract, potassium iodide and mixtures thereof. Mucolytics useful in the present invention include, but, are not restricted to the group consisting of acetylcycsteine, ambroxol, bromhexine and mixtures thereof. Analgesic, antipyretic and anti-inflammatory agents useful in the present invention include, but, are not restricted to the group consisting of acetaminophen, aspirin, diclofenac, diflunisal, etodolac, fenoprofen, flurbiprofen, ibuprofen, ketoprofen, ketorolac, nabumetone, naproxen, piroxicam, caffeine and mixtures thereof. Local anesthetics useful in the present invention include, but, are not restricted to the group consisting of lidocaine, benzocaine, phenol, dyclonine, benzonotate and mixtures thereof.

Solvents

The un-ionized form of the pharmaceutical active is maintained using a selected group of solvents. The solvent portion of compositions of the present invention comprises from about 60% to about 99.975%, preferably from 70% to about 99% and most preferably from about 85% to about 98% by weight of the composition.

The solvent of the present invention is normally liquid at ambient or room temperatures. It is water-soluble or water-miscible. Solvents of the present invention are preferably selected from the group consisting of propylene glycol, ethanol, poly(ethylene glycol) or PEG, propylene carbonate, diethylene glycol monoethyl ether, poloxamer, glycofurol, glycerol, and mixtures thereof. Propylene glycol and ethanol is particularly preferred. There are mixtures of these solvents that are particularly preferred for certain product forms of the present invention. For example, if the product form is an elixir, liquid capsule or liquid containing lozenge, the solvent is a combination of propylene glycol, ethanol, and PEG. If the product form is a spray, the solvents is a combination of propylene glycol, ethanol, PEG and usually propylene carbonate. The level of each solvent that makes up these mixtures is partially dependent on aesthetic benefits sought by the formulator. Most preferable are anhydrous forms of the above solvents.

Reducing Agents

The addition of reducing agents has been found to have a beneficial chemical stabilizing effect on the actives comprising the present invention. This phenomena surprisingly takes place where the active is in different phase than the reducing agent. For example, where the active is soluble in a non-polar enviroment or phase of the composition, the reducing agent selected should be a polar phase, such as water. Therefore, despite being in separate phases, the chemical stability of the active is still positively impacted. The same stability benefit is not observed when the active and the reducing agent are cosoluble in the solvent. Therefore, the reducing agents useful in the composition depend on the active selected and its solubility.

Reducing agents are substances that have a lower redox potential than the drug or adjuvant that they are intended to protect against oxidation. Thus reducing agents are more readily oxidized than the drug or adjuvant and are effective in the presence of oxidizing agents. See W. Lund, The Pharmaceutical DODEX, 12th Edition, p.290, The Pharmaceutical Press, 1994, incorporated herein by reference. Reducing agents of the present have a electrode potential value. This is defined by the Nernst equation and practically measured using standard electrochemical reference cells. The resulting values are therefore called the Standard Electrode Potential, of E₀ as measured in volts of (V). Comparing standard electrode potentials for different substances can be used to assess the effectiveness of different reducing agents; see Wells, Pharmaceutical Preformulation, Ellis Horwood Limited Publishing, 1988, pp. 168-172; incorporated herein by reference. The reducing useful in the present invention have E value greater than about −0.11 9 V, preferably from about −0.1 19 V to +0.250 V. Preferred reducing agents are selected from the group consisting of the salts of meta bisulfite and bisulfite, including their sodium and potassium salts, dithiothreitol, thiourea, sodium thiosulphate, thioglycolic acid, terbuty hydroquinone (TBHQ), acetyl cysteine, hydroquinone and mixtures thereof.

The level of reducing agents useful in the present invention is from about 0.005% to 1.000%, preferably from about 0.500% to about 0.050%, and most preferably from about 0.100% to about 0.010% by weight of the composition.

Optional Ingredients

Water may be used in compositions of the present invention. In the present invention the maximum level of water is about 10%, preferably from about 1% to about 10% more preferably from 5% to about 10% and most preferably from about 5% to about 8% by weight of the composition.

Ingredients normally associated with cold and influenza treatment medicines can be used with the pharmaceutical actives disclosed herein. Such ingredients are disclosed in U.S. pat. No. 5,196,436, incorporated herein by reference. Additionally, the following ingredients may be used in the present invention:

Buffers and mixtures of buffering agents, including basic buffers as single components with pKa of from 8 to 11, include triethanolamine, tromethamine, salts of amino acids, including alkaline salts of glycine, glycylglycine, glutamine or other amino acids, alkaline salts of phosphate, carbonate and mixtures thereof. The buffers provide compositional resistance to pH changes upon dilution of the composition with saliva within the range of 8 to 10.

Sweeteners, including aspartame, saccharin and its salts, Sucralose™ (sold by the McNeil Specialty Products Co., New Brunswick, N..J.); Prosweet™ (sold by the Virginia Dare Extract Co., New York, N.Y.); Magnasweet™ (sold by MAFCO Worldwide Corp., Licorice Division, Camden, N..J.); ammonium glycyrrhizinate, its salts, Talin™ (Thaumatin) and its diluted products, such as Talin GA90, (sold by the Talin Food Company, Birkenhead, England); and Acesulfame K, and mixtures thereof.

Flavorants, include anise, oil of peppermint, oil of clove, eucalyptus, lemon, lime, honey lemon, red fruit, mint, grapefruit, orange, cherry cola and mixtures thereof.

Sensory agents. Also useful herein are sensory agents selected from the group consisting of coolants, salivating agents, warming agents. Preferably these agents are present in the compositions at a level of from about 0.001% to about 10%, preferably from about 0.1% to about 1%, by weight of the composition.

Suitable cooling agents and warming agents include carboxamides, menthols, thymol, camphor, capsicum, phenol, eucalyptus oil, benzyl alcohol, salicyl alcohol, ethanol, clove bud oil, and hexylresorcinol, ketals, diols, and mixtures thereof. Preferred warming agents include thymol, camphor, capsicum, phenol, benzyl alcohol, salicyl alcohol, ethanol, clove bud oil, and hexylresorcinol, nicotinate esters such as benzyl nicotinate, ketals, diols, and mixtures thereof.

Preferred coolants are the paramenthan carboxyamide agents such as N-ethyl-p-menthan-3-carboxamide (WS-3 supplied by Sterling Organics), taught by U.S. pat. No. 4,136,163, issued Jan. 23, 1979, to Watson et al., which is incorporated herein by reference in its entirety. Preferred coolants are the paramenthan carboxyamide agents such as N-ethyl-p-menthan-3-carboxamide. Another preferred paramenthan carboxyamide agent is N,2,3-trimethyl-2-isopropylbutanamide, known as “WS-23”, and mixtures of WS-3 and WS-23.

Additional preferred coolants are selected from the group consisting of menthol, 3- 1-menthoxypropane-1,2-diol, known as TK-10 supplied by Takasago Perfumery Co., Ltd., Tokyo, Japan, menthone glycerol acetal known as MGA, manufactured by Haarmann and Reimer, menthyl lactate known as Frescolat( manufactured by Haarmann and Reimer, and mixtures thereof.

Additonal cooling agents include cyclic sulphones and sulphoxides and others, all of which are described in U.S. pat. No. 4,032,661, issued Jun. 28, 1977, to Rowsell et al., which is herein incorporated by reference.

The terms “menthol” and “menthyl” as used herein include dextro- and levoratotory isomers of these compounds and racemic mixtures thereof.

TK-10 is described in detail in U.S. pat. No. 4,459,425, issued Jul. 10, 1984 to Amano et al. and incorporated herein by reference.

Salivating agents of the present invention include Jambu® manufactured by Takasago Perfumery Co., Ltd., Tokyo, Japan. METHOD OF USE

In terms of the methods of delivery of the active, it is generally accepted that oral mucosal delivery inside the mouth must be targeted to the sub-lingual region in order to achieve a very rapid therapeutic effect; see D. Harris and J. R. Robinson, Drug Delivery via the Mucus Membranes of the Oral Cavity, Journal of Pharmaceutical Sciences 81: 1, 1992. Such dosage forms are designed to be placed under the tongue, on the floor of the mouth, and held there for some extended time. The inventors have found, however, that a large increase in bioavailability with very rapid absorption can be achieved when the subject compositions are placed against any of the mucosal membranes of the mouth, even onto the tongue and swallowed. The form of the invention is a liquid elixir solution. It is intended to be applied to any of the mucosal membranes within the mouth. This can be achieved using a medicine dropper that is calibrated to indicate the proper amount to be administered, and squirting the elixir onto the tongue prior to swallowing. The elixir can be atomized into mouth and throat and then swallowed. It can be encapsulated into some sort of shell which makes it portable and convenient to transport and administer without having to measure the quantity of liquid elixir. Examples of encapsulation shell includes hard candies as are used for lozenges, gelatin, or starch-based shells. The elixir may be packaged into a small, disposable vial which can readily be opened and squirted into the mouth, the entire vial containing exactly one therapeutic dose. Typical dosage forms of the composition of the present invention contain no more than about 3 ml., preferable from about 0.2 ml. to about 3 ml.

One preferred form is to encapsulate the liquid into a shell of hard candy or gelatin. The shell containing substances to pretreat the mucosa and thereby enhance the absorption of the active from the liquid center. The pretreatment occurs by sucking or chewing the shell material, and the advantage is gained by separating in time the treatment of the mucosa, which occurs first, followed by the presentation of the active to be absorbed. Examples of substances for pretreatment of the mucosal membranes are membrane penetration enhancers that are commonly known in the art, examples including menthol, peppermint oil, surfactants such as polysorbate 80 or poloxamer. Another example of a mucosal membrane pretreatment are buffers as listed above, which would precondition salivary micro environment pH in the range of 8 to 11.

Liquid Elixir

| | | % Comp. |
|---|---|---|
| Item # | Material | (w/w) |
| 1 | Propylene Glycol | 80.764 |
| 2 | Ethanol (100%) | 9.000 |
| 3 | Purified Water | 5.000 |
| 4 | Sodium Metabisulfite | 0.050 |
| 5 | Sodium Saccharin | 0.650 |
| 6 | Peppermint Flavorant | 2.000 |
| 7 | Acesulfame K¹ | 0.450 |
| 8 | Takasago 10² | 0.100 |
| 9 | Methone Glycerine Acetal | 0.300 |
| 10 | Ethyl Methane Carboxamide | 0.070 |
| 11 | Monoammonium Glycyrrhizinate | 0.150 |
| 12 | Dextromethorphan Base | 1.466 |
| | Total | 100.000 |

1 Acesulfame K available Nutrinova Inc Company of Somerset,N..J.-08873, USA 2 TK 10 available from Takasago Company Of Rockleigh, N..J.-07657, USA

Add a portion of Ethanol to the active (Dextromethorphan base) and solid sweetening agents (Sucralose, Monoammonium glycyrrizinate) and continuously mix at low heat (30° C.). To this vessel add the Propylene Glycol and liquid sweeteners (Pro-sweet Liquid K). Add the reducing agent (meta bisulfite) and water together and mix until uniform. Add the mixture to the vessel and mix for about 2 hours time. Add a premix of flavorants and colorants in the remaining portion of ethanol, and add to the vessel containing the nearly completed solution. Mix until a homogenous solution is obtained. Allow the composition to reside in the mixing vessel, open to the atmosphere for about 10 minutes. Filter the composition through a US # 100 mesh sieve (product density=1.07 g/ml.). Fill into amber glass bottles, and cap with an integrated cap/calibrated medicine dropper assembly.

About 1.5 grams of the elixir dropped onto the tongue and then swallowed. Dextromethorphan is rapidly absorbed into the blood.

Liquid Elixir

| | | % Comp. |
|---|---|---|
| Item # | Material | (w/w) |
| 1 | Dextromethorphan Base | 2.055 |
| 2 | Ethanol (100%) | 10.000 |
| 3 | Propylene Glycol | 83.275 |
| 4 | Sodium Meta Bisulfite | 0.010 |
| 5 | Tnethanolamine | 3.740 |
| 6 | Sucralose | 0.150 |
| 7 | Pro-Sweet Liquid K | 0.700 |
| 8 | Monoammonium Glycyrrhizinate | 0.050 |
| 9 | Flavorant | 0.015 |
| 10 | Colorant | 0.005 |
| | Total | 100.000 |

Add a portion of Ethanol to the active (Dextromethorphan base) and solid sweetening agents (Sucralose, Monoammonium glycyrrizinate) and continuously mixed at low heat (30° C.). To this vessel add the Propylene Glycol, liquid sweeteners (Pro-sweet Liquid K), and buffer (Triethanolamine, a liquid). Add the metabisulfide and mix until all materials are in solution, about 2 hours time. Add a premix of flavorants and colorants in the remaining portion of ethanol, and add to the vessel containing the nearly completed solution. Mix until a homogenous solution is obtained. Allow the composition to reside in the mixing vessel, open to the atmosphere for about 10 minutes. Mix until a homogenous solution is obtained, and filter through a US # 100 mesh sieve (product density=1.07 g/ml.). Fill into amber glass bottles, and cap with an intergrated cap/calibrated medicine dropper assembly.

About 1.0 ml. of the elixir dropped onto the tongue and then swallowed. Dextromethorphan is rapidly absorbed into the blood.

Liquid Spray

| | | % Comp. |
|---|---|---|
| Item # | Material | (w/w) |
| 1 | Dextromethorphan Base | 3.425 |
| 2 | Thioglycerol | 0.050 |
| 3 | Propylene Glycol | 95.335 |
| 5 | Sucralose | 0.300 |
| 6 | Pro-Sweet Liquid K | 0.700 |
| 7 | Monoammonium Glycyrrhizinate | 0.050 |
| 8 | Flavorant | 0.015 |
| 9 | Colorant¹ | 0.005 |
| | Total | 100.000 |

¹Green Shade CSL-15689 obtained from the Warner Jenkins Co., St. Louis MO, U.S.A.

1. Green Shade CSL-15689 obtained from the Warner Jenkins Co., St. Louis, MO., USA.

Add a portion of propylene glycol to the active (Dextromethorphan base) and solid sweetening agents (Sucralose, Monoammonium glycyrrizinate) and continuously mixed at low heat (30° C.). To this vessel add the additional propylene glycol and liquid sweeteners (Pro-sweet Liquid K). Add the thioglycerol and mix until all materials are in solution, about 2 hours time. Add a premix of flavorants and colorants in the remaining portion of ethanol, and add to the vessel containing the nearly completed solution. Mix until a homogenous solution is obtained. Allow the composition to reside in the mixing vessel, open to the atmosphere for about 10 minutes. Mix until a homogenous solution is obtained, and filter through a US # 100 mesh sieve (product density=1.075 g/ml.). Fill into manually operated atomization pump and bottle. An example is manufactured by Calmar-Albert GmbH,the Mistette Mark II fitted with a 16 mm high viscosity head assembly which delivers 0.2 ml./actuation.

Three individual actuations are sprayed into the mouth. Dextromethorphan is rapidly absorbed into the blood, and during spraying some portion of the sprayed liquid contacts the throat area, providing the additional benefit such as numbing of the irritated cough receptors there.

Liquid Spray

| | | % Comp. |
|---|---|---|
| Item # | Material | (w/w) |
| 1 | Dextromethorphan Base | 3.425 |
| 2 | Ethanol (100%) | 5.350 |
| 3 | Propylene Glycol | 41.315 |
| 4 | Propylene Carbonate | 40.000 |
| 5 | Triethanolamine | 3.740 |
| 6 | Thioglycerol | 0.050 |
| 7 | Sucralose | 0.300 |
| 8 | Pro-Sweet Liquid K | 0.700 |
| 9 | Monoammonium Glycyrrhizinate | 0.050 |
| 10 | Flavorant | 0.015 |
| 11 | Purified Water | 5.000 |
| 12 | Potassium Metabisulfite | 0.050 |
| 13 | Colorant | 0.005 |
| | Total | 100.000 |

Add a portion of Ethanol to the active (Dextromethorphan base) and solid sweetening agents (Sucralose, Monoammonium glycyrrizinate) and continuously mixed at low heat (30° C.). To this vessel add the additional Propylene Carbonate and Propylene liquid sweeteners (Pro-sweet Liquid K) reducing agent and buffer (Triethanolamine, a liquid). Mix until all materials are in solution, about 2 hours time. Allow the composition to reside in the mixing vessel, open to the atmosphere for about 10 minutes. Prepare a premix of flavorants and colorants in the remaining portion of ethanol, and add to the vessel containing the nearly completed solution. Mix until a homogenous solution is obtained, and filter through a US # 100 mesh sieve (product density=1.075 g/ml. Fill into manually operated atomization pump and bottle. An example is manufactured by Calmar-Albert GmbH, the Mistette Mark II fitted with a 16 mm high viscosity head assembly.

Three individual actuations are sprayed into the mouth. Dextromethorphan is rapidly absorbed into the blood, and during spraying some portion of the sprayed liquid contacts the throat area, providing the additional benefit such as numbing of the irritated cough receptors there.

Liquid Centered Lozenge

| | | % Comp. |
|---|---|---|
| Item # | Material | (w/w) |
| 1 | Dextromethorphan Base | 2.055 |
| 2 | Ethanol (100%) | 2.000 |
| 3 | Purified Water | 5.000 |
| 4 | Propylene Glycol | 84.825 |
| 5 | Sodium Metabisulfite | 0.050 |
| 6 | Sucralose | 0.300 |
| 7 | Pro-Sweet Liquid K | 0.700 |
| 8 | Monoammonium Glycyrrhizinate | 0.050 |
| 9 | Flavorant | 0.015 |
| 10 | Colorant | 0.005 |
| | Total | 100.000 |

Add a portion of Ethanol to the active (Dextromethorphan base) and solid sweetening agents (Sucralose, Monoammonium glycyrrizinate) and continuously mixed at low heat (30° C.). To this vessel add the Propylene Glycol, and liquid sweeteners (Pro-sweet Liquid K). Mix until all materials are in solution, about 2 hours time. Mix until a homogenous solution is obtained. Prepare a premix of flavorants and colorants in the remaining portion of ethanol, sodium metabisulfite and water, and add to the vessel containing the nearly completed solution. Allow the composition to reside in the mixing vessel, open to the atmosphere for about 10 minutes. Mix until a homogenous solution is obtained, and filter through a US # 100 mesh sieve (product density=1.07 g/ml.). Make individual filled lozenges containing about 1.0 ml. of liquid per lozenge by a commonly used method such as extrusion

A person places a liquid filled lozenge into the mouth and sucks on the lozenge until the liquid fill is released. Some cough relief is obtained through the action of sucking on the shell of the lozenge. When the liquid center is released, dextromethorphan is rapidly absorbed into the blood.

Liquid Centered Lozenge

| | | % Comp. |
|---|---|---|
| Item # | Material | (w/w) |
| 1 | Dextromethorphan Base | 2.055 |
| 2 | Ethanol (100%) | 2.000 |
| 3 | Purified Water | 5.000 |
| 4 | Propylene Glycol | 84.875 |
| 5 | Sodium Metabisulfite | 0.050 |
| 6 | Sucralose | 0.300 |
| 7 | Pro-Sweet Liquid K | 0.700 |
| 8 | Monoammonium Glycyrrhizinate | 0.050 |
| 9 | Flavorant | 0.015 |
| 10 | Colorant | 0.005 |
| | Total | 100.000 |

Add a portion of Ethanol to the active (Dextromethorphan Base) and solid sweetening agents (Sucralose, Monoammonium glycyrrizinate) and continuously mixed at low heat (30° C.). To this vessel add the Propylene Glycol, and liquid sweeteners (Pro-sweet Liquid K). Prepare an aqueous premix of sodium metabisulfite and add to the vessel. Mix until all materials are in solution, about 2 hours time. Prepare a premix of flavorants and colorants in the remaining portion of ethanol, and add to the vessel containing the nearly completed solution. Allow the composition to reside in the mixing vessel, open to the atmosphere for about 10 minutes. Mix until a homogenous solution is obtained, and filter through a US # 100 mesh sieve (product density=1.07 g/ml.). Make individual filled lozenges containing about 1.0 ml. of liquid per lozenge by a commonly used method such as extrusion

A person places a liquid filled lozenge into the mouth and sucks until the liquid fill is released. Some cough relief is obtained through the action of sucking on the shell of the lozenge. When the liquid center is released, dextromethorphan is rapidly absorbed into the blood, and relief from coughing is obtained within 10 minutes time.

Liquid Elixir

| | | % Comp. |
|---|---|---|
| Items # | Material | (w/w) |
| 1 | Dextromethorphan Base | 2.055 |
| 2 | Pseudoephedrine Base | 4.593 |
| 3 | Ethanol (100%) | 10.000 |
| 4 | Propylene Glycol | 5.000 |
| 5 | Triethanolamine | 3.740 |
| 6 | Sucralose | 0.150 |
| 7 | Pro-Sweet Liquid K | 0.700 |
| 8 | Monoammonium Glycyrrhizinate | 0.050 |
| 9 | Flavorant | 0.015 |
| 10 | Colorant | 0.005 |
| 11 | Sodium Metabisulfite | 0.050 |
| | Total | 100.000 |

Add a portion of Ethanol to the active (Dextromethorphan base & Pseudoephedrine base) and solid sweetening agents (Sucralose, Monoammonium glycyrrizinate) and continuously mixed at low heat (30° C.). To this vessel add the bulk of the propylene glycol, liquid sweeteners (Pro-sweet Liquid K), and buffer (Triethanolamine, a liquid). Mix until all materials are in solution, about 2 hours time. Prepare a premix of flavorants and colorants in the remaining portions of propylene glycol and ethanol,as well as the sodium metabisulfite and add to the vessel containing the nearly completed solution. Mix until a homogenous solution is obtained, and filter through a US # 100 mesh sieve (product density=1.07 g/ml.). Fill into amber glass bottles, and cap with an integrated cap/calibrated medicine dropper assembly.

About 1.0 ml. of the elixir dropped onto the tongue and then swallowed.

Liquid Elixir

| | | % Comp. |
|---|---|---|
| Items # | Material | (w/w) |
| 1 | Chlorpheniramine Base | 0.263 |
| 2 | Pseudoephedrine Base | 4.593 |
| 3 | Ethanol (100%) | 10.000 |
| 4 | Propylene Glycol | 5.000 |
| 5 | Sucralose | 0.150 |
| 6 | Pro-Sweet Liquid K | 0.700 |
| 7 | Monoammonium Glycyrrhizinate | 0.050 |
| 8 | Flavorant | 0.015 |
| 9 | Colorant | 0.005 |
| 10 | Sodium Bisulfite | 0.050 |
| | Total | 100.000 |

Add a portion of Ethanol to the actives (Chlorpheniramine base & Pseudoephedrine base) and solid sweetening agents (Sucralose, Monoammonium glycyrrizinate) and continuously mixed at low heat (30° C.). To this vessel add the bulk of the propylene glycol, liquid sweeteners (Pro-sweet Liquid K), sodium bisulfite and buffer (Triethanolamine, a liquid). Mix until all materials are in solution, about 2 hours time. Prepare a premix of flavorants and colorants in the propylene glycol and remaining portion of ethanol, and add vessel containing the nearly completed solution. Mix until a homogenous solution is obtained, and filter through a US # 100 mesh sieve (product density=1.07 g/ml.). Fill into amber glass boffles, and cap with an integrated cap/calibrated medicine dropper assembly.

About 1.0 ml. of the elixir dropped onto the tongue and then swallowed. Chlorpheniramine & pseudoephedrine is rapidly absorbed into the blood.

Liquid Elixir

| | | % Comp. |
|---|---|---|
| Items # | Material | (w/w) |
| 1 | Acetoaminophen | 27.169 |
| 2 | Dextromethorphan Base | 1.195 |
| 2 | Pseudoephednne Base | 2.671 |
| 3 | Ethanol (100%) | 10.000 |
| 4 | Propylene Glycol | 52.034 |
| 5 | Polyvinyl pyrrolidone² | 2.170 |
| 6 | Triethanolamme | 3.740 |
| 7 | Sucralose | 0.150 |
| 8 | Pro-Sweet Liquid K | 0.700 |
| 9 | Monoatnmonium Glycyrrhizinate | 0.050 |
| 10 | Flavorant | 0.015 |
| 11 | Colorant | 0.005 |
| 12 | Sodium Metabisulfite | 0.100 |
| | Total | 100.000 |

¹Carbowax Sentry Polyethylene available from Union Carbide, Mooretown NJ, U.S.A.
²PVP-K17PF available from BASF Corp.

1. Carbowax Sentry Polyethylene available from Unoin Carbide, Mooretown N..J., USA.

2. PVP-K17PF available from BASF Corp.

Dissolve Dextromethorphan Base and Pseudoephedrine Base in portion of alcohol to make a premix. In separate container heat propylene glycol to about 70° C. Once all material is melted and in clear liquid form add Acetoamonophen and continue to heat to 110-120° C. with continuous mixing. Remove heat once liquid is clear. Cool it to room temperature. Add the mixture to the Dextromethorphan and Pseudoephedrine. Also add liquid sweetener (Pro-sweet Liquid K) and buffer (Triethanolamine).

Mix until all materials are in solution. Prepare a premix of flavorants and colorants in the remaining portion of alcohol and sodium metabisulfite, and add to the vessel containing the nearly completed solution. Allow the composition to reside in the mixing vessel, open to the atmosphere for about 10 minutes. Mix until homogeneous and filter through a US #100 mesh sieve. Fill in a amber glass bottles, and cap with an integrated cap/calibrated medicine dropper assembly. About 1.84 grams of the elixir is dropped onto the tongue and then swallowed.

Liquid Elixir

| | | % Comp. |
|---|---|---|
| Items # | Material | (w/w) |
| 1 | Ethanol (100%) | 88.484 |
| 2 | Water | 10.00 |
| 3 | Dextromethorphan Base | 1.466 |
| 4 | Meta Bisulphite | 0.05 |
| | Total | 100.000 |

Dissolve Dextromethorphan Base in portion of alcohol to make a premix. In separate container heat water and meta Bisulfite to about 70° C. Mix until uniform and cool to room temperature. Add this mixture to the Dextromethorphan Base.

Mix until all materials are in solution. Add the remaining portion of alcohol and sodium metabisulfite to the vessel containing the nearly completed solution. Allow the composition to reside in the mixing vessel, open to the atmosphere for about 10 minutes. Mix until homogeneous and filter through a US #100 mesh sieve. Fill in a amber glass bottles, and cap with an integrated cap/calibrated medicine dropper assembly. About 1.84 grams of the elixir is dropped onto the tongue and then swallowed.

Liquid Elixir

| | | % Comp. |
|---|---|---|
| Items # | Material | (w/w) |
| 1 | Ethanol (100%) | 88.484 |
| 2 | Water | 10.00 |
| 3 | Dex base | 1.466 |
| 4 | Metabisulphite | 0.05 |
| 5 | Aesthetics package¹ | 4.000 |
| | Total | 100.000 |

1. see above examples

Dissolve Dextromethorphan Base in portion of alcohol to make a premix. In separate container heat water and meta Bisulfite to about 70° C. Mix until uniform and cool to room temperature. Add this mixture to the Dextromethorphan Base.

Mix until all materials are in solution. Add the remaining portion of alcohol, sodium metabisulfite and the aesthetics package to the vessel containing the nearly completed solution. Allow the composition to reside in the mixing vessel, open to the atmosphere for about 10 minutes. Mix until homogeneous and filter through a US #100 mesh sieve. Fill in a amber glass bottles, and cap with an integrated cap/calibrated medicine dropper assembly. About 1.84 grams of the elixir is dropped onto the tongue and then swallowed.

chewable soft gellatin capsules

| | | % Comp. |
|---|---|---|
| Items # | Material | (w/w) |
| 1 | Propylene Glycol | 35.109 |
| 2 | Glycerine | 10.000 |
| 3 | Dextromethorphan Base | 1.100 |
| 4 | Acetoaminophen Base | 32.500 |
| 5 | Pseudoephedrine Base | 2.458 |
| 6 | Polyvinyl pyrrolidone | 4.170 |
| 7 | Aesthetics package¹ | 4.000 |
| 8 | Water | 10.000 |
| 9 | Potassium Metabisulfite | 0.050 |
| | Total | 100.000 |

1. see above examples

Dissolve Dextromethorphan Base in portion of alcohol to make a premix. In separate container heat water and meta Bisulfite to about 70° C. Add acetoamonophen and continue to heat to 110-120° C. with continuous mixing. Remove heat once liquid is clear. Cool it to room temperature. Add the mixture to the Dextromethorphan and PseudoephedrineMix until uniform and cool to room temperature. Mix until all materials are in solution. Add the remaining portion of alcohol, polyvinyl pyrrolidone, sodium metabisulfite and the aesthetics package to the vessel containing the nearly completed solution. Allow the composition to reside in the mixing vessel, open to the atmosphere for about 10 minutes. Mix until homogeneous and filter through a US #100 mesh sieve. Fill chewable soft gellatin capsules using the above formulation. Said gelatin capsules are available from the trade by companies such as R. P. Scherer, of St. Petersberg, Fla. About 1.84 grams of the elixir is delivered to the mouth by mastication of the capsule(s) and then swallowed.

chewable soft gellatin capsules

| | | % Comp. |
|---|---|---|
| Items # | Material | (w/w) |
| 1 | Propylene Glycol | 74.950 |
| 2 | Glycerine | 10.000 |
| 3 | Dextromethorphan Base | 1.100 |
| 4 | Aesthetics package¹ | 4.000 |
| 5 | Water | 10.000 |
| 9 | Potassium Metabisulfite | 0.050 |
| | Total | 100.000 |

1. see above examples

Dissolve Dextromethorphan Base in portion of alcohol to make a premix. In separate container heat water and Meta Bisulfite to about 70° C. Remove heat once liquid is clear. Cool it to room temperature. Add the mixture to the Dextromethorphan. Mix until uniform and cool to room temperature. Mix until all materials are in solution. Add the remaining portion of alcohol and the aesthetics package to the vessel containing the nearly completed solution. Allow the composition to reside in the mixing vessel, open to the atmosphere for about 10 minutes. Mix until homogeneous and filter through a US #100 mesh sieve. Fill chewable soft gellatin capsules using the above formulation. Said gelatin capsules are available from the trade by companies such as R. P. Scherer, of St. Petersberg, Fla. About 1.84 grams of the elixir is delivered to the mouth by mastication of the capsule(s) and then swallowed.

We claim:

1. A liquid composition having improved stability comprising a pharmaceutical active, solvent to solubilize said active, and a reducing agent to improve said active stability in said composition.

2. An oral composition having improved stability comprising a pharmaceutical active, solvent to solubilize said active, and a reducing agent to improve said active stability in said composition.

3. The composition according to claim 1 wherein the reducing agent has an E⁰ value of greater than −0.1 19 V.

4. The composition according to claim 3 wherein the reducing agent has an E⁰ value from about −0.1 19 V to +0.250 V.

5. The composition according to claim 4 wherein the reducing agent is selected from the group consisting of the salts of meta bisulfite and bisulfite, including their sodium and potassium salts; dithiothreitol; thiourea; sodium thiosulphate; thioglycolic acid; terbuty hydroquinone (TBHQ); acetyl cysteine; hydroquinone and mixtures thereof.

6. The composition according to claim 5 wherein the reducing agent comprises from about 0.005% to 1.000% of the composition.

7. The composition according to claim 6 wherein the reducing agent comprises from about 0.100% to about 0.01% by weight of the composition.

8. A composition according to claim 5 comprising a pharmaceutical active in an hydrophilic, water-miscible, anhydrous solvent wherein the pharmaceutical active in its un-ionized form has a percent solubility value in the solvent at ambient temperature that is equal to or greater than 0.075% and the pharmaceutical active is in it free, un-ionized form as a monomolecular dispersion in the solvent and said water.

9. The composition according to claim 8 wherein the pharmaceutical actives have a molecular weight of less than 500 grams per mole, is capable of being ionized when in an aqueous solvent and has an octanol-water partition coefficient when in the un-ionized form of at least 100.

10. The composition according to claim 9 wherein the pharmaceutical actives are selected from the group consisting of antitussives, antihistamines, non-sedating antihistamines, decongestants, expectorants, analgesic mucolytics, antipyretic anti-inflammatory agents, local anesthetics and mixtures thereof.

11. The composition according to claim 10 wherein the concentration of pharmaceutical actives in the solvent is less than or equal to 125% of the percent solubility value of said active.

12. The composition according to claim 11 wherein the pharmaceutical active is present in the solvent at a level from about 0.075% to about 25.0% by weight of the composition.

13. The composition according to claim 12 wherein the pharmaceutical active is present in the solvent at a level from about 0.28% to 10.0%.

14. The composition according to claim 13 wherein the solvent comprises from about 60% to about 99.975% by weight of the composition.

15. The composition according to claim 14 wherein the comprises from about 70% to about 99% by weight of the composition.

16. The composition according to claim 15 wherein the solvent comprises from about 85% to about 98% by weight of the composition.

18. The composition according to claim 15 wherein the solvent is hydrophilic, water-miscible, and anhydrous selected from the group consisting propylene glycol, ethanol, poly(ethylene glycol) or PEG, propylene carbonate, diethylene glycol monoethyl ether, poloxamer, glycofurol, glycerol and mixtures thereof.

19. A method for treating respiratory illnesses using the composition of claim 2 wherein the method comprises oral administration of said composition having a total dosage volume of no more than 3.0 mls.

20. The method according to claim 19 wherein the composition is placed against any of the mucosal membranes of the mouth.
